(19) United States
(12) Patent Application Publication
(10) Pub. No.: US 2026/0331283 A1
(43) Pub. Date: Oct. 22, 2026

(54) Portable, Low-Cost Medical Diagnostic System Utilizing Deep Learning for Cancer Detection

(71) Applicants: Samaira Mehta, Santa Clara, CA (US); Aadit Mehta, Santa Clara, CA (US)

(72) Inventors: Samaira Mehta, Santa Clara, CA (US); Aadit Mehta, Santa Clara, CA (US)

(21) Appl. No.: 19/181,312

(22) Filed: Apr. 16, 2025

(51) Int. Cl. G16H 50/20; G06T 5/60; G06T 5/70; G06T 7/00; G06T 7/11; G06T 7/136; G06V 10/24; G06V 10/26; G06V 10/36; G06V 10/764; G06V 10/82; G06V 10/94; G06V 20/69; G16H 10/60; G16H 30/40

(52) U.S. Cl. CPC G16H 50/20; G06T 5/60; G06T 5/70; G06T 7/0012; G06T 7/11; G06T 7/136; G06V 10/26; G06V 10/36; G06V 10/764; G06V 10/82; G06V 10/955; G06V 20/695; G06V 20/698; G16H 30/40; G06T 2200/24; G06T 2207/10056; G06T 2207/20021; G06T 2207/20081; G06T 2207/20084; G06T 2207/20092; G06T 2207/30096; G06V 10/242; G06V 2201/03; G16H 10/60

(57) ABSTRACT

The present invention relates to a portable, low-cost medical diagnostic system utilizing deep learning-based artificial intelligence for the automated classification of cancer subtypes from histopathological images. The system is implemented on an embedded computing platform, specifically a Raspberry Pi-based architecture, enabling real-time analysis without requiring continuous internet connectivity or high-performance computational infrastructure. The invention features a structured image processing pipeline, including patch extraction, tile generation, tissue content filtering, and artifact removal, to enhance classification accuracy and optimize diagnostic outcomes. A deep convolutional neural network (DLCNN), based on a fine-tuned VGG-16 architecture, is trained to classify ovarian cancer subtypes with 95% accuracy, generating classification results with confidence scores and visual heatmap overlays. The system supports dual deployment modes, operating either as a standalone edge-based device for offline usage in resource-constrained environments or as a cloud-integrated platform for remote diagnostics.

### BACKGROUND OF THE INVENTION

### Field of Invention

The present invention relates to a portable, low-cost medical diagnostic system utilizing deep learning algorithms for cancer detection and classification. Specifically, the invention provides a standalone diagnostic framework implemented on a Raspberry Pi-based embedded system, capable of analyzing histopathological images to identify and classify cancer subtypes with high accuracy. The system is designed for both cloud-based and edge-computing deployments, enabling decentralized, infrastructure-independent diagnostics, particularly in resource-limited healthcare settings. By leveraging machine learning techniques, digital pathology, and automated image analysis, the invention enhances diagnostic accuracy while reducing reliance on traditional laboratory infrastructure. While particularly suited for ovarian cancer subtype classification, the system is adaptable for broader histopathological diagnostics across various cancer types.

### BRIEF SUMMARY OF THE INVENTION

The present invention provides a portable, low-cost medical diagnostic system that utilizes deep learning algorithms for the automated detection and classification of cancer subtypes from histopathological images. Implemented on a Raspberry Pi-based embedded system, the invention offers a self-contained, infrastructure-independent diagnostic tool that can operate without internet connectivity or reliance on high-performance computing. The system processes whole-slide histopathology images through a structured pipeline involving patch extraction, tile generation, and tissue content filtering to enhance classification accuracy. A deep convolutional neural network (CNN), specifically an adapted VGG-16 architecture, is trained to classify ovarian cancer subtypes with a reported accuracy of 95%. The diagnostic results, including subtype classification and confidence scores, are displayed through a custom graphical user interface, with options for local storage, USB-based data transfer, or cloud-based accessibility via a web platform.

By integrating edge-computing capabilities, the system enables real-time cancer diagnosis in resource-limited settings where traditional pathology infrastructure is unavailable. The device is entirely self-powered using a rechargeable battery pack, ensuring usability in remote or underserved areas. Additionally, the invention supports cloud deployment for broader accessibility, allowing medical professionals to upload histopathology images via a web interface and receive diagnostic insights. This dual deployment strategy-cloud-based and edge-based-ensures that the system can function effectively in both well-equipped medical institutions and decentralized healthcare environments. While designed primarily for ovarian cancer diagnosis, the framework is adaptable for other histopathological applications, demonstrating a scalable and cost-effective solution for AI-driven cancer diagnostics.

### BRIEF DESCRIPTION OF THE FIGURES

FIG. 1 illustrates the complete workflow of the portable AI-driven histopathological diagnostic system, encompassing image acquisition and preprocessing (FIG. 1.101), structured patch extraction and tile generation (FIG. 1.103), tissue content filtering and artifact removal (FIG. 1.105), deep learning-based subtype classification (FIG. 1.107), confidence scoring and heatmap generation (FIG. 1.109), result output and storage (FIG. 1.111), and deployment strategies for both edge-based and cloud-based diagnostics (FIG. 1.113).

FIG. 2: A visual overview of the end-to-end OVision workflow, including dataset preprocessing, deep learning model training, and final deployment to edge and cloud platforms.

FIG. 3: A schematic representation of the dataset preparation pipeline, illustrating how whole slide images are transformed into filtered image tiles for training and evaluation.

FIG. 4: Representative examples of histopathological image patches labeled with ovarian cancer subtypes by pathologists for supervised model training.

FIG. 5: The adapted VGG-16 deep learning network architecture used for multi-class classification of ovarian cancer subtypes.

FIG. 6: User interface of the OVision web platform, showing the three-step process for remote cancer subtype detection.

FIG. 7: Physical design of the final Raspberry Pi-powered portable medical device, with integrated battery and display for standalone cancer classification.

FIG. 8: Illustration of the step-by-step usage process of the handheld diagnostic device using local image upload and on-device classification.

FIG. 9: Normalized confusion matrix depicting the classification performance of the deep learning model across all ovarian cancer subtypes.

FIG. 10 displays the receiver operating characteristic (ROC) curves and area under the curve (AUC) metrics for the deep learning classifier, illustrating high accuracy in distinguishing ovarian cancer subtypes.

FIG. 11 illustrates the deployment of the OVision diagnostic system in a clinical or field setting, showing its integration with microscopy and local diagnostic workflows.

### DETAILED DESCRIPTION

The present invention relates to a portable, low-cost medical diagnostic system designed to perform automated detection and classification of cancer subtypes from histopathological images using deep learning-based artificial intelligence (AI) models. The system is implemented on an embedded computing platform, specifically a Raspberry Pi-based architecture, allowing for real-time diagnostic capability independent of high-performance computational resources or continuous internet connectivity. The invention is designed to address critical diagnostic accessibility challenges, particularly in resource-constrained environments, by providing a self-contained, infrastructure-independent histopathology analysis tool that can be deployed in rural clinics, mobile healthcare units, and field hospitals, where access to trained pathologists and laboratory-grade diagnostic equipment is limited or unavailable. The invention provides dual-mode functionality, supporting both standalone edge computing-based operation and cloud-integrated deployment, thereby enhancing its adaptability across different clinical environments.

The hardware configuration of the system comprises an embedded computing module, a graphical user interface (GUI), a power management system, a data input interface, and an optional microscope integration unit. The embedded computing module is built upon a Raspberry Pi 4 microcomputer, selected for its optimal balance of computational efficiency, power consumption, and affordability. The processor architecture includes a quad-core ARM Cortex-A 72 CPU, with a minimum of 2GB RAM, and an optimized TensorFlow Lite execution environment to support efficient deep learning inference with minimal latency. The LCD display, preferably ranging from 2.5 inches to 5 inches, provides a real-time graphical interface for displaying classification results and facilitating user interactions with the device. The power management system includes a high-capacity rechargeable lithium-ion battery pack, ensuring that the device remains operational in locations lacking stable electricity. The data input interface supports multiple modes of image acquisition, including USB-based image transfer, direct digital pathology integration, and optional microscope-mounted camera connectivity, allowing users to process whole-slide biopsy images from various medical imaging sources. The device further supports physical output capabilities, such as USB storage for result transfer and an optional printing module for immediate generation of diagnostic reports.

The algorithmic processing module is designed to process histopathological biopsy images using a structured, multi-stage deep learning pipeline. The image preprocessing workflow consists of patch extraction, tile generation, tissue content filtering, and artifact removal to enhance classification accuracy. The patch extraction process involves segmenting whole-slide biopsy images at 20× magnification, ensuring that diagnostically relevant tissue regions are isolated while excluding background areas. The tile generation process further subdivides the patches into smaller regions, which are then analyzed for tissue content thresholding, eliminating non-informative image regions. A computational thresholding method, based on file size and pixel density, is employed to ensure that only tiles containing at least 50% tissue content are retained for processing, reducing false-positive and false-negative diagnostic errors.

The invention employs a deep convolutional neural network (DLCNN) model, specifically a modified VGG-16 architecture, to classify cancer subtypes from histopathological images. The neural network architecture consists of multiple convolutional layers, batch normalization layers, max-pooling layers, and fully connected layers, optimized for high-accuracy feature extraction and classification of oncological tissue samples. The trained model provides a probabilistic classification output across five major ovarian cancer subtypes, namely high-grade serous carcinoma (SC), low-grade serous carcinoma (LC), clear cell carcinoma (CC), endometrioid carcinoma (EC), and mucinous carcinoma (MC). The model has been validated on a curated histopathology dataset, demonstrating an overall classification accuracy of 95%, with high sensitivity and specificity across all cancer subtypes. The classification results are displayed in real-time via the GUI, and include primary subtype prediction, ranked secondary predictions, confidence scores, and heatmap visualizations to improve interpretability and diagnostic decision-making.

The invention supports dual deployment modes: cloud-based mode and edge-based mode. In cloud-based mode, the trained deep learning model is hosted on a virtual private server (VPS), enabling medical professionals to upload histopathology images via a secure web interface and receive classification results remotely. The cloud platform, deployed on DigitalOcean or equivalent cloud infrastructure, ensures high availability, scalability, and data security, with encrypted patient data transmission and HIPAA-compliant storage mechanisms. This mode is particularly advantageous for medical institutions, research laboratories, and clinical training centers, where remote AI-driven diagnostic analysis can augment existing pathology workflows.

In edge-based mode, the device functions as a fully standalone diagnostic system, executing deep learning inference locally on the Raspberry Pi without reliance on cloud connectivity. This mode is optimized for deployment in regions with limited internet access, such as rural healthcare centers, field hospitals, mobile diagnostic units, and disaster relief zones. The embedded computing architecture is designed for low-power consumption, ensuring sustained diagnostic capability even in environments with unstable power sources. Local storage capabilities enable diagnostic results to be saved on the device, transferred via USB, or printed directly for record-keeping and clinical documentation.

A key advantage of the invention is its ability to automate cancer subtype classification, significantly reducing diagnostic turnaround time and reliance on highly trained pathologists. The system enables quantitative analysis of tumor heterogeneity, allowing for tile-by-tile classification within a single biopsy sample, which provides a more detailed diagnostic perspective than traditional whole-slide pathology assessment. This capability enhances precision oncology applications, as oncologists can develop personalized treatment plans based on the specific histological composition of the tumor. The system's high-speed inference capability, with classification results generated in under 10 seconds, further enhances its clinical utility in time-sensitive diagnostic scenarios.

The invention's modular architecture allows for scalability and extension to other cancer types, beyond ovarian cancer. The deep learning framework can be retrained and fine-tuned with additional histopathological datasets to support classification of other malignancies, such as breast cancer, lung cancer, and prostate cancer. The preprocessing pipeline, including patch extraction, tile generation, and tissue content filtering, can be adapted to different histological staining techniques and tissue morphologies, ensuring broad applicability in AI-assisted oncological diagnostics.

By integrating edge computing, embedded AI processing, and cloud-based deep learning inference, the present invention offers a transformative solution for democratizing cancer diagnostics. The device provides a cost-effective, portable, and highly accurate alternative to traditional pathology workflows, bridging the gap between state-of-the-art AI-powered diagnostics and real-world clinical accessibility. As machine learning capabilities continue to evolve, future iterations of the system may incorporate additional predictive biomarkers, molecular profiling techniques, and real-time clinical decision support, further expanding its impact on cancer diagnosis and treatment planning. The invention's dual deployment strategy ensures that advanced oncological diagnostic tools are accessible to both high-resource medical institutions and low-resource healthcare settings, marking a significant advancement in AI-driven precision oncology.

A patentability analysis was conducted in view of prior art, specifically Indian Patent IN202041040783, which discloses a breast cancer detecting device using Raspberry Pi and machine learning techniques. While both the prior art and the present invention utilize Raspberry Pi as an embedded computing platform for cancer detection, the present invention introduces substantial technical advancements and novel features that differentiate it from existing solutions.

The prior art primarily focuses on breast cancer detection and describes a real-time application device utilizing a charge-coupled device (CCD) camera and an LCD for visualization, wherein machine learning techniques classify breast histopathology images into benign and malignant categories. The present invention, by contrast, is specifically designed for multi-class histopathological subtype classification in ovarian cancer, employing a structured image preprocessing pipeline, deep convolutional neural networks (DLCNNs), and a dual-mode (edge and cloud-based) deployment strategy to enhance accessibility and diagnostic performance.

One key distinction is the advanced image preprocessing pipeline incorporated in the present invention, which includes patch extraction, tile generation, and tissue content filtering to ensure the removal of non-informative regions and artifacts. This structured approach is not disclosed in IN202041040783, where the method of histopathological image processing is not detailed. By segmenting whole-slide images into diagnostically relevant tiles, the present invention significantly improves classification accuracy and computational efficiency, reducing false-positive and false-negative rates compared to raw image classification.

Furthermore, the deep learning architecture utilized in the present invention represents a significant technological advancement over the prior art. The disclosed system employs a fine-tuned VGG-16-based DLCNN, optimized for multi-class ovarian cancer subtype classification, achieving 95% accuracy—a level of diagnostic precision that is not reported in the prior art. In contrast, the prior art does not specify which machine learning algorithms are utilized, nor does it disclose any subtype-specific classification methodology. The present invention's AI model has been carefully trained and validated on a curated ovarian cancer histopathology dataset, ensuring robust and clinically relevant classification results.

Additionally, the deployment flexibility of the present invention surpasses that of the prior art. While IN202041040783 describes a handheld breast cancer detection device, it does not mention offline functionality or cloud integration. The present invention, however, is designed to operate in both edge-based and cloud-based modes, allowing for real-time, offline inference on a Raspberry Pi microcomputer or remote AI-based diagnosis via a cloud-hosted model. This dual deployment strategy ensures continuous operability, particularly in low-resource settings where internet access is unavailable—a feature that is not taught or suggested in the prior art.

Moreover, the present invention introduces interpretability enhancements not disclosed in IN202041040783, including probabilistic confidence scoring and heatmap visualization via grad-CAM overlays, enabling pathologists to verify AI-based predictions and assess tissue regions contributing to classification results. These features provide critical decision-support tools for medical professionals and significantly improve AI-assisted diagnostic reliability.

Therefore, while the prior art generally discloses a Raspberry Pi-based cancer detection system, it fails to teach or suggest the structured preprocessing pipeline, deep learning-based multi-class subtype classification, dual deployment framework, or advanced AI interpretability enhancements disclosed in the present invention. These novel and non-obvious advancements establish a clear distinction from existing solutions, providing a technically superior, more accurate, and versatile histopathological diagnostic system for oncological applications beyond the scope of the prior art.

### DETAILED DESCRIPTION OF FIGURES

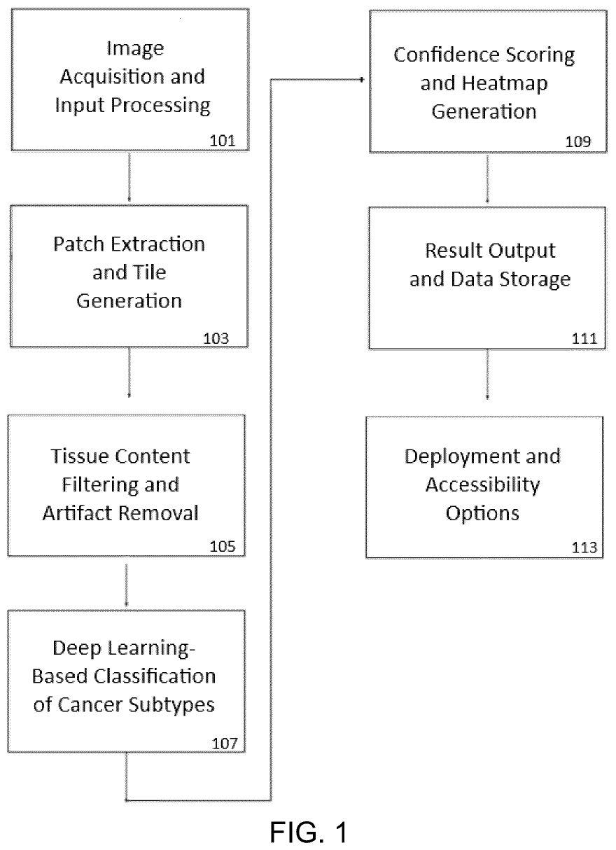

FIG. 1.101—Image Acquisition and Input Processing The first step in the diagnostic workflow involves acquiring histopathological biopsy images from various sources, including digital pathology slides, microscope-mounted cameras, or external storage devices such as USB drives. If the image is captured using a digital microscope, the system receives raw whole-slide images (WSIs) at a standard magnification level, typically 20×, ensuring optimal resolution for diagnostic analysis. The image input processing module then performs preliminary format standardization, converting files into a compatible structure for deep learning-based classification. Metadata extraction is also performed to ensure that critical patient or imaging details are retained for subsequent analysis.

FIG. 1.103—Patch Extraction and Tile Generation Once the image has been standardized, the system performs patch extraction, which involves segmenting the whole-slide image into non-overlapping patches of predefined dimensions. Each extracted patch undergoes tile generation, further subdividing the image into smaller image tiles at 224×224 pixel resolution, aligning with the pretrained deep learning model's input format. The system ensures that extracted patches represent diverse tissue regions by covering tumor cells, stromal tissue, and other diagnostically relevant structures to enhance classification accuracy.

FIG. 1.105—Tissue Content Filtering and Artifact Removal

After tile generation, the system applies a tissue content filtering algorithm to eliminate image tiles containing excessive background or non-informative regions. This process utilizes a computational threshold based on pixel density and file size, discarding tiles with less than 50% tissue content to reduce false-positive or false-negative results. Additionally, artifact removal techniques are applied to correct image distortions, staining variations, and scanner-induced noise that may interfere with accurate deep learning inference.

FIG. 1.107—Deep Learning-Based Classification of Cancer Subtypes

The preprocessed histopathological image tiles are fed into a deep convolutional neural network (DLCNN) based on a modified VGG-16 architecture. The trained model performs multi-class classification, predicting the likelihood of the image belonging to one of five ovarian cancer subtypes: high-grade serous carcinoma (SC), low-grade serous carcinoma (LC), clear cell carcinoma (CC), endometrioid carcinoma (EC), or mucinous carcinoma (MC). The classification process is optimized for real-time execution on a Raspberry Pi microcomputer, with TensorFlow Lite inference acceleration ensuring rapid, low-latency diagnostic predictions.

FIG. 1.109—Confidence Scoring and Heatmap Generation

Following classification, the system generates probabilistic confidence scores for each subtype, allowing medical professionals to assess the reliability of the predictions. The confidence scoring algorithm assigns a weighted probability distribution across all possible subtypes, with the highest-ranking subtype designated as the primary classification result. Additionally, a grad-CAM (Gradient-weighted Class Activation Mapping) heatmap visualization is generated, overlaying a color-coded diagnostic confidence map onto the original histopathological image to highlight key tissue regions contributing to the prediction.

FIG. 1.111—Result Output and Data Storage

The classification results, including primary subtype prediction, ranked secondary predictions, confidence scores, and heatmap overlays, are displayed via a graphical user interface (GUI) on the embedded Raspberry Pi system. The results can be stored locally for on-device retrieval, exported via USB transfer, or transmitted to a centralized cloud database for remote accessibility. An optional automated report generation module formats the classification findings into a standardized pathology report, facilitating seamless integration with electronic medical record (EMR) systems.

FIG. 1.113—Deployment and Accessibility Options The final step in the workflow involves deploying the system across different operational environments, including edge-based standalone usage and cloud-based remote analysis. In edge computing mode, the system functions as a fully self-contained diagnostic tool, running deep learning inference locally on the Raspberry Pi without requiring an internet connection. In cloud-based mode, histopathological images are uploaded to a remote server, where classification is performed using a cloud-hosted DLCNN model, with results accessible via a secure web-based interface. The device is optimized for deployment in rural clinics, mobile healthcare units, and resource-limited medical facilities, enabling AI-driven histopathological diagnostics in settings where traditional pathology infrastructure is unavailable.

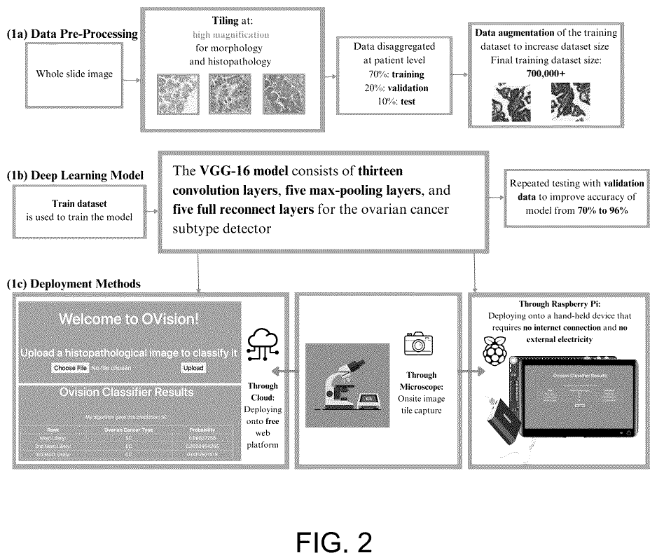

FIG. 2 presents a comprehensive visualization of the full OVision system workflow. It begins with data preprocessing, wherein histopathology slides are collected and digitized. Using 20× magnification, patches are extracted from these whole-slide images, tiled into smaller units, and filtered to exclude tiles containing more than 50% white space. Data augmentation techniques, such as rotations and color adjustments, are applied to enhance dataset variability. The dataset is then partitioned into training, validation, and test sets on a per-patient basis, thereby avoiding data leakage and ensuring that model evaluation is unbiased and generalizable. The next segment illustrates the iterative model training process, where a deep learning model is trained using the curated image dataset to optimize classification accuracy. The final segment displays the deployment architecture, showing how the trained model is exported to both a cloud server (accessible through a web interface) and a compact, battery-powered Raspberry Pi-based handheld device. A microscope with camera capability is shown as an optional component, enabling direct local image acquisition aligned with the model's training specifications. The full framework, including code and documentation, is made publicly accessible through the GitHub repository: https://github.com/samairamehta/OVision-Framework.

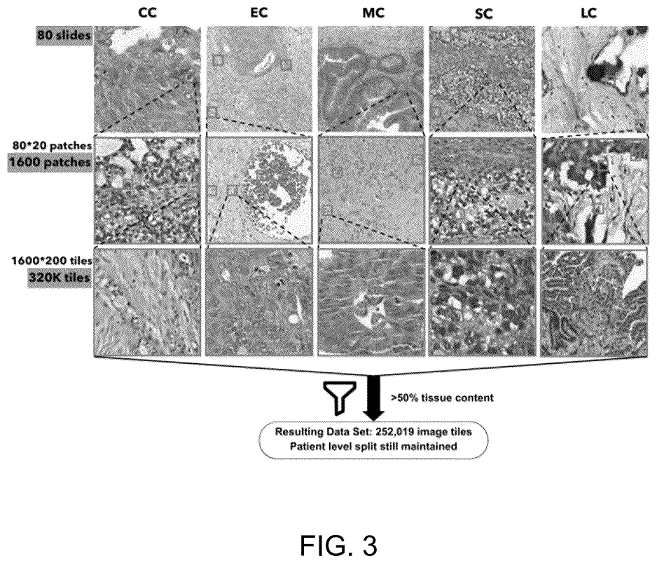

FIG. 3 illustrates the end-to-end pipeline used to generate the dataset used for model training and evaluation. The process begins with acquisition of 80 whole-slide histopathological images, each representing a distinct ovarian cancer case. A patient-level split is applied to divide data into training (70%), validation (20%), and testing (10%) subsets to ensure model generalizability. From each slide, 20 non-overlapping patches are extracted at 20× magnification. Each patch is further subdivided into 200 tiles of 224×224 pixels. A filtering process eliminates tiles containing less than 50% tissue, resulting in a final curated dataset of 252,019 high-quality image tiles. These filtered tiles are then used for deep learning model training and performance evaluation.

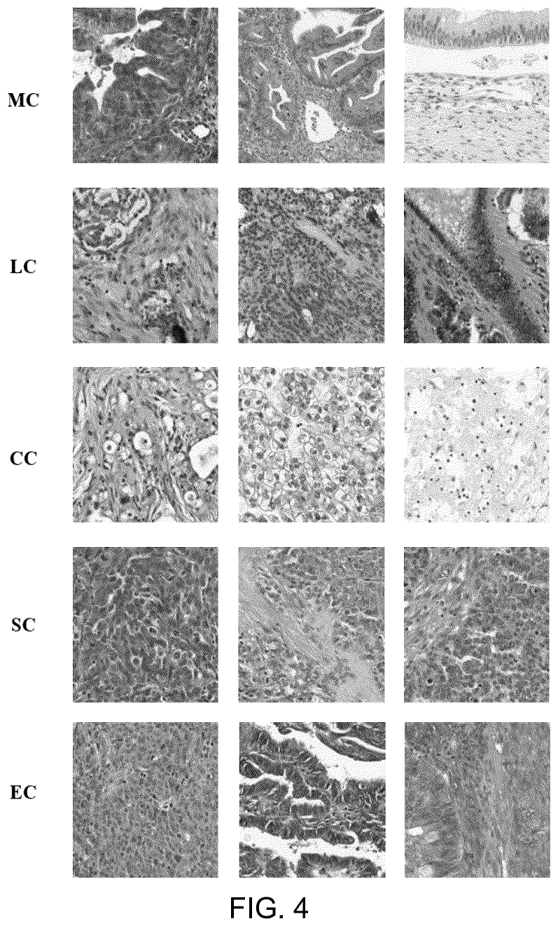

FIG. 4 displays representative image patches from the training dataset, each labeled with a specific ovarian cancer histologic subtype by expert pathologists. The visualized categories include: Mucinous Carcinoma (MC), Low Grade Serous Carcinoma (LC), Clear Cell Carcinoma (CC), High Grade Serous Carcinoma (SC), and Endometrioid Carcinoma (EC). These manually annotated samples are used to train the classification model, helping it learn unique visual patterns associated with each subtype. The example tiles reflect the morphological heterogeneity present across ovarian cancer specimens, supporting the model's ability to distinguish between closely related subtypes.

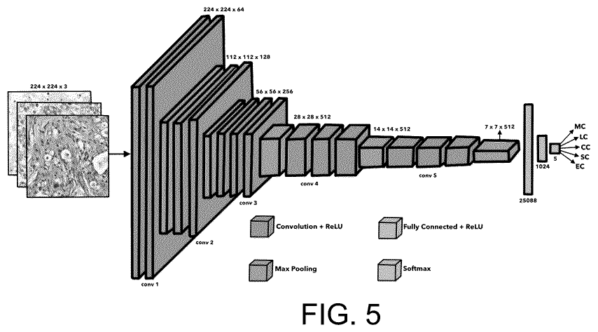

FIG. 5 provides a schematic diagram of the VGG-16 convolutional neural network architecture as adapted for ovarian cancer subtype classification. The network comprises 13 convolutional layers with increasing depth, interspersed with max-pooling layers to downsample spatial dimensions. The convolutional base is followed by fully connected dense layers, culminating in a 5-class softmax output layer for predicting the subtype category. The architecture is pre-trained on ImageNet and subsequently fine-tuned on the ovarian cancer dataset, with modifications including additional dense layers and customized hyperparameters to enhance classification performance within a resource-constrained environment.

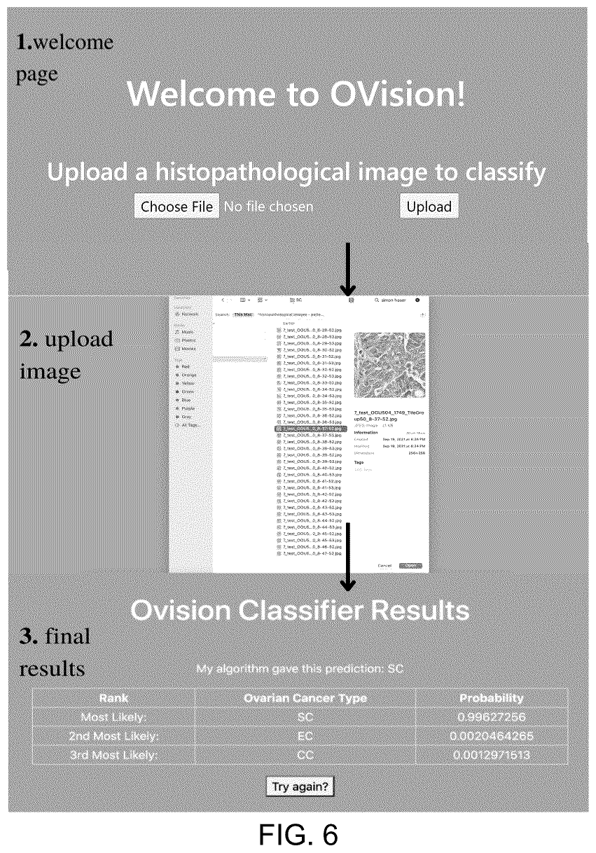

FIG. 6 presents the user interface of the OVision web-based diagnostic platform. Designed for accessibility, the interface guides the user through a three-step process: (1) visiting the platform, (2) selecting a histopathological image file for upload, and (3) initiating the upload to receive classification results. The backend cloud server processes the image using the trained deep learning model and returns the most probable cancer subtype, along with a ranked list of alternative predictions and their confidence scores. This platform democratizes access to AI-driven diagnostics in any internet-enabled healthcare setting.

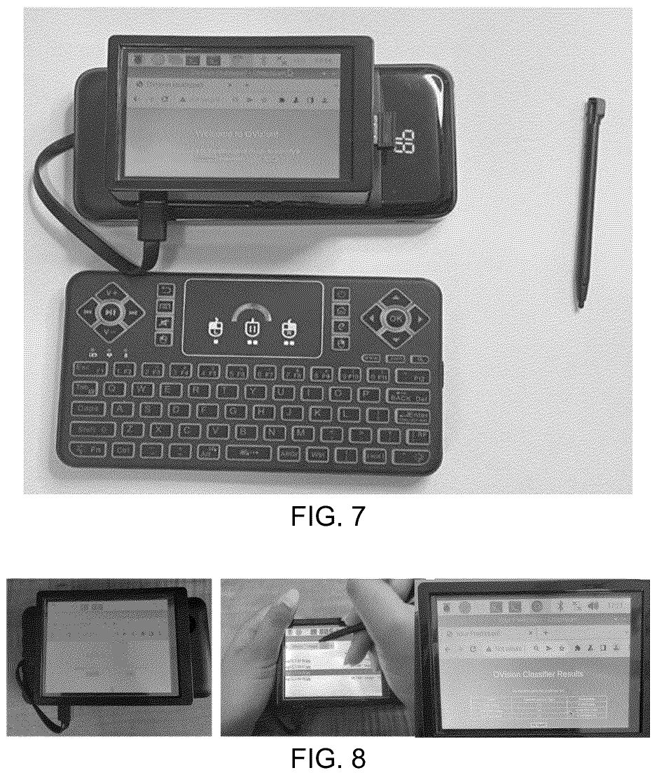

FIG. 7 illustrates the physical configuration of the final OVision handheld diagnostic device. The compact 2.5-inch by 3-inch device integrates a Raspberry Pi 4 microcomputer, LCD display, rechargeable battery pack, and optional mini keyboard for interaction. The deep learning model is pre-loaded and optimized for execution on TensorFlow Lite, allowing the device to perform offline, real-time cancer subtype classification without the need for network connectivity. USB ports are included for image input, and classification results are presented on the device's screen in under 10 seconds.

FIG. 8 details the streamlined operational workflow for using the handheld device. In Step 1, the user inserts a USB storage device containing digital pathology images into the unit. In Step 2, the GUI prompts the user to select the file for analysis. In Step 3, the device processes the image and outputs the predicted ovarian cancer subtype and confidence scores. This intuitive three-click process is optimized for non-specialist users and is especially valuable in remote clinics or mobile health units.

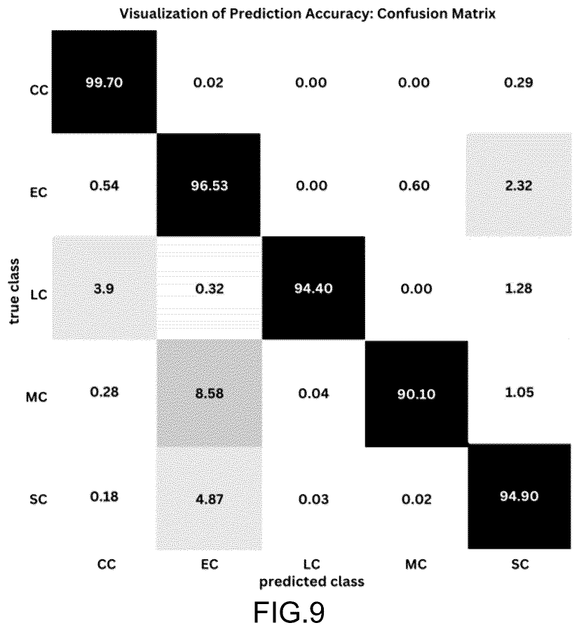

FIG. 9 depicts the normalized confusion matrix for the trained deep learning model, showing the distribution of correct and incorrect predictions across the five ovarian cancer subtypes. Each diagonal entry represents the percentage of correct predictions for a specific subtype, while off-diagonal entries indicate misclassifications. High diagonal values confirm the model's ability to accurately distinguish between closely related subtypes. The matrix also demonstrates balanced performance, with minimal confusion among classes, thereby validating the classifier's robustness and reliability.

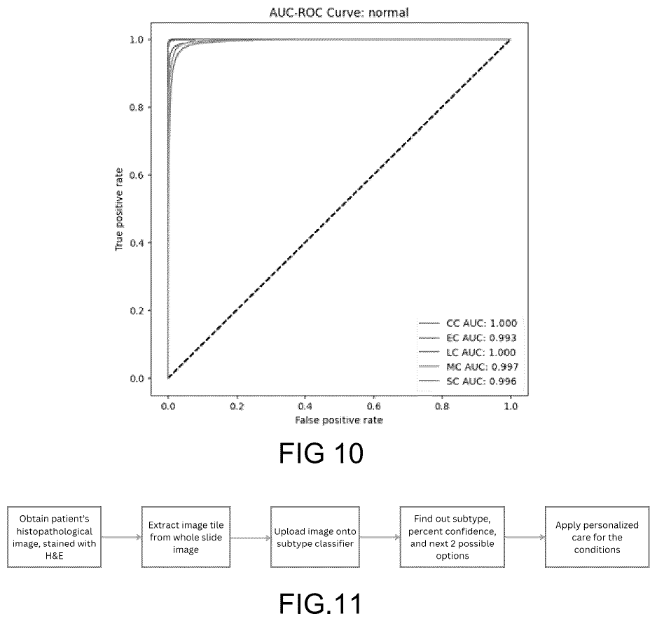

FIG. 10 presents the ROC curve and corresponding AUC values for each of the five ovarian cancer subtypes classified by the trained DLCNN model. The ROC curve plots the true positive rate (sensitivity) against the false positive rate (1-specificity) across various thresholds, providing a holistic view of model performance. AUC values for all subtypes range from 0.90 to 1.00, placing the model in the “excellent” diagnostic category based on standard evaluation metrics. This confirms the classifier's robustness in differentiating between histologically similar subtypes and underscores its clinical utility in automated ovarian cancer subtype detection.

FIG. 11 provides a contextual depiction of the OVision system in a practical deployment scenario, such as a point-of-care clinic or remote diagnostic setup. The image demonstrates how a microscope captures digital histopathological images, which are then transferred to either the Raspberry Pi-powered handheld device or the cloud-based interface for classification. This visual emphasizes the system's ability to operate independently of centralized infrastructure, highlighting features such as offline AI inference, portability, and compatibility with on-site imaging tools. The figure reinforces the intended use of OVision in low-resource or decentralized medical environments, enabling equitable access to precision oncology diagnostics.

What is claimed is:

1-20. (canceled)

21. A portable edge-computing histopathological image analysis system, comprising:
an image acquisition interface configured to receive histopathological image data from at least one of a microscope-mounted camera, a removable storage device, or a direct digital pathology interface;
an embedded computing device comprising a Raspberry Pi 4 microcomputer having a quad-core ARM Cortex-A 72 processor, a minimum of 2GB local volatile memory, and non-transitory local storage storing processor-executable instructions;
a local display interface comprising an LCD display operatively coupled to the embedded computing device and configured to present histopathological classification output and user interface controls without requiring a remote display server;
a local result storage interface configured to persistently store classification outputs on the embedded computing device or an attached removable storage medium without requiring continuous network connectivity; and
a rechargeable lithium-ion battery-based power management circuit configured to supply power to the embedded computing device, the image acquisition interface, and the local display interface during image acquisition and classification operations in the absence of an external power source;
wherein the processor-executable instructions, when executed by the Raspberry Pi 4 microcomputer, cause the system to perform an ordered histopathological image-processing pipeline comprising:
(a) receiving the histopathological image data through the image acquisition interface;
(b) partitioning the histopathological image data into a plurality of non-overlapping patches at approximately 20× magnification and generating image tiles of 224×224 pixels from the patches;
(c) computing, for each generated image tile, a tissue-content metric based on pixel density, file size, or both, quantifying a proportion of non-background tissue within that tile;
(d) discarding image tiles for which the tissue-content metric indicates less than approximately fifty percent tissue content, thereby excluding background, artifact, and non-diagnostic image regions from subsequent processing;
(e) retaining image tiles for which the tissue-content metric indicates at least approximately fifty percent tissue content;
(f) applying artifact removal to the retained image tiles to suppress image distortions, staining variations, and scanner-induced noise that would reduce classification reliability;
(g) executing, locally on the Raspberry Pi 4 microcomputer using a TensorFlow Lite-optimized deep convolutional neural network stored on the non-transitory local storage, without transmitting the retained image tiles to any remote server and without requiring continuous internet connectivity, a tile-by-tile inference on each retained image tile to produce a tile-level ovarian cancer subtype classification output;
(h) aggregating the tile-level ovarian cancer subtype classification outputs across all retained image tiles to generate a specimen-level classification output comprising at least one primary ovarian cancer subtype prediction and at least one quantitative confidence score; and
(i) presenting, through the local display interface, the specimen-level classification output together with a heatmap visualization identifying retained image tiles associated with the highest classification confidence scores; and
wherein discarding the image tiles for which the tissue-content metric indicates less than approximately fifty percent tissue content in step (d) reduces a peak volatile-memory utilization and a per-image inference time of the Raspberry Pi 4 microcomputer, such that the ordered histopathological image-processing pipeline of steps (a) through (i) executes within the minimum 2GB local volatile memory and generates the specimen-level classification output in under ten seconds per histopathological image file.

22. The system of claim 21, wherein the tissue-content metric of step (c) is computed based on pixel density and file size of each generated image tile, and wherein tiles for which said metric indicates less than approximately fifty percent tissue content are discarded prior to execution of the TensorFlow Lite-optimized deep convolutional neural network.

23. The system of claim 21, wherein the heatmap visualization of step (i) is a Gradient-weighted Class Activation Mapping (Grad-CAM) overlay superimposed on the histopathological image data, the Grad-CAM overlay indicating the geographic locations of retained image tiles associated with elevated ovarian cancer subtype classification confidence scores, thereby providing an explainable localization output for medical professional review.

24. The system of claim 21, wherein the TensorFlow Lite-optimized deep convolutional neural network is stored entirely within the non-transitory local storage of the embedded computing device and all of steps (a) through (i) are performed locally on the Raspberry Pi 4 microcomputer without transmitting histopathological image tiles or classification outputs to any remote server.

25. The system of claim 21, wherein the processor-executable instructions further cause the embedded computing device to output a ranked list of secondary ovarian cancer subtype predictions in addition to the primary ovarian cancer subtype prediction, wherein each ranked secondary prediction is associated with a respective secondary quantitative confidence score.

26. The system of claim 21, wherein the processor-executable instructions further cause the embedded computing device to persist the specimen-level classification output to at least one of: the non-transitory local storage of the embedded computing device, a removable storage medium coupled through a USB interface, or a portable storage device operatively connected to the local result storage interface.

27. The system of claim 21, wherein the processor-executable instructions further cause the embedded computing device to generate a structured pathology report comprising the specimen-level classification output, the quantitative confidence score, imaging acquisition metadata, and the heatmap visualization of step (i), and to store said structured pathology report in a standardized format on the non-transitory local storage.

28. The system of claim 21, wherein the artifact removal of step (f) comprises identifying and excluding from the retained image tile set at least one of: image tiles exhibiting image distortions exceeding a distortion threshold, tissue-free background regions, regions exhibiting non-uniform staining density indicative of staining variation, or image tiles containing scanner-induced noise failing a minimum signal quality criterion.

29. The system of claim 21, wherein the processor-executable instructions further cause the embedded computing device to automatically set a high-priority review flag and generate a local alert when the quantitative confidence score for the primary ovarian cancer subtype prediction satisfies a predetermined confidence threshold or subtype-specific review rule, thereby enabling expedited clinical follow-up without requiring network transmission.

30. The system of claim 21, wherein the local display interface presents both a primary ovarian cancer subtype classification label and the heatmap visualization that graphically delineates histopathological image regions of the retained image tiles having the highest contribution to the specimen-level ovarian cancer subtype prediction, and wherein the classification results are generated and displayed in under ten seconds per histopathological image file selected through the user interface of the embedded computing device.

31. The system of claim 21, wherein the rechargeable lithium-ion battery-based power management circuit is configured to sustain continuous operation of the embedded computing device for at least eight hours of combined image acquisition and local classification operations under typical load conditions, thereby supporting uninterrupted diagnostic sessions in decentralized clinical settings lacking reliable external power infrastructure.

32. The system of claim 21, wherein the TensorFlow Lite-optimized deep convolutional neural network is a fine-tuned VGG-16 architecture pre-trained on ImageNet and subsequently fine-tuned on a curated ovarian cancer histopathology dataset, the fine-tuned VGG-16 architecture comprising thirteen convolutional layers and a five-class softmax output layer corresponding to the five ovarian cancer subtypes of high-grade serous carcinoma, low-grade serous carcinoma, clear cell carcinoma, endometrioid carcinoma, and mucinous carcinoma, and optimized for inference on the ARM Cortex-A72 processor using TensorFlow Lite, wherein all inference steps are performed locally on the Raspberry Pi 4 microcomputer without transmitting image data to a remote server.

33. The system of claim 21, wherein the image acquisition interface further comprises a microscope-mounted camera adapter configured to mechanically couple a digital image sensor to an ocular lens port of a light microscope, and wherein the processor-executable instructions cause the embedded computing device to capture histopathological image data directly from said microscope-mounted camera adapter at approximately 20× magnification.

34. The system of claim 21, wherein the processor-executable instructions further cause the embedded computing device to apply staining-variation correction to the retained image tiles to reduce staining variations in the retained image tile set, as part of the artifact removal of step (f), prior to execution of the TensorFlow Lite-optimized deep convolutional neural network in step (g).

35. A computer-implemented method for local histopathological image classification on a portable embedded computing device, the method comprising:
receiving histopathological image data through an image acquisition interface operatively coupled to the portable embedded computing device, the portable embedded computing device comprising a Raspberry Pi 4 microcomputer having a quad-core ARM Cortex-A72 processor and a minimum of 2GB RAM;
partitioning the histopathological image data into a plurality of non-overlapping patches at approximately 20× magnification and generating image tiles of 224×224 pixels from the patches using the ARM Cortex-A72 processor;
computing, for each image tile, a tissue-content metric based on pixel density, file size, or both, representing a proportion of non-background tissue content within that tile;
discarding image tiles for which the tissue-content metric indicates less than approximately fifty percent tissue content;
retaining image tiles for which the tissue-content metric indicates at least approximately fifty percent tissue content;
applying artifact removal to the retained image tiles to suppress image distortions, staining variations, and scanner-induced noise;
executing, locally on the ARM Cortex-A72 processor using a TensorFlow Lite-optimized deep convolutional neural network stored on non-transitory local storage of the portable embedded computing device, without transmitting the retained image tiles to any remote server and without requiring continuous internet connectivity, a tile-by-tile inference on the retained image tiles to generate tile-level ovarian cancer subtype classification outputs;
aggregating the tile-level ovarian cancer subtype classification outputs to generate a specimen-level ovarian cancer subtype classification comprising a primary ovarian cancer subtype prediction and a quantitative confidence score; and
displaying, on a local display interface of the portable embedded computing device, the specimen-level ovarian cancer subtype classification together with a heatmap visualization identifying retained image tiles associated with elevated classification confidence scores;
wherein discarding the image tiles for which the tissue-content metric indicates less than approximately fifty percent tissue content reduces a peak volatile-memory utilization and a per-image inference time of the Raspberry Pi 4 microcomputer, such that the method executes within the minimum 2GB RAM and generates the specimen-level ovarian cancer subtype classification in under ten seconds per histopathological image file.

36. The method of claim 35, wherein the heatmap visualization is a Gradient-weighted Class Activation Mapping (Grad-CAM) overlay superimposed on the histopathological image data, visually delineating retained image tile locations associated with the highest ovarian cancer subtype classification confidence scores.

37. The method of claim 35, further comprising storing the specimen-level ovarian cancer subtype classification, the quantitative confidence score, and the heatmap visualization in a locally generated structured pathology report on non-transitory local storage of the portable embedded computing device, without transmitting said report to an external server.

38. The method of claim 35, further comprising automatically generating a high-risk classification flag and a local alert on the portable embedded computing device when the quantitative confidence score for the primary ovarian cancer subtype prediction satisfies a predetermined high-risk threshold, the local alert being generated locally without requiring network transmission to an external system.

39. The method of claim 35, wherein executing the TensorFlow Lite-optimized deep convolutional neural network locally comprises executing a fine-tuned VGG-16 architecture stored entirely on non-transitory local storage of the portable embedded computing device on the ARM Cortex-A72 processor, wherein TensorFlow Lite optimization reduces the model's memory and computational requirements to enable inference within the RAM constraints of the Raspberry Pi 4 microcomputer, achieving classification results in under ten seconds per histopathological image file selected through the user interface of the portable embedded computing device.

40. A non-transitory computer-readable storage medium storing processor-executable instructions that, when executed by a Raspberry Pi 4 microcomputer having a quad-core ARM Cortex-A72 processor and a minimum of 2GB RAM, cause the Raspberry Pi 4 microcomputer to perform operations comprising:
receiving histopathological image data through an image acquisition interface;
partitioning the histopathological image data into a plurality of non-overlapping patches at approximately 20× magnification and generating image tiles of 224×224 pixels from the patches;
computing, for each image tile, a tissue-content metric based on pixel density, file size, or both, representing a proportion of non-background tissue content;
discarding image tiles for which the tissue-content metric indicates less than approximately fifty percent tissue content;
retaining image tiles for which the tissue-content metric indicates at least approximately fifty percent tissue content;
applying artifact removal to the retained image tiles to suppress image distortions, staining variations, and scanner-induced noise;
executing, locally on the ARM Cortex-A72 processor using a TensorFlow Lite-optimized deep convolutional neural network stored on non-transitory local storage, without transmitting the retained image tiles to any remote server and without requiring continuous internet connectivity, a tile-by-tile inference on the retained image tiles to generate tile-level ovarian cancer subtype classification outputs;
aggregating the tile-level ovarian cancer subtype classification outputs to generate a specimen-level ovarian cancer subtype classification comprising a primary ovarian cancer subtype prediction and a quantitative confidence score; and
displaying, on a local display interface operatively coupled to the Raspberry Pi 4 microcomputer, the specimen-level ovarian cancer subtype classification together with a heatmap visualization identifying retained image tiles associated with elevated classification confidence scores;
wherein discarding the image tiles for which the tissue-content metric indicates less than approximately fifty percent tissue content reduces a peak volatile-memory utilization and a per-image inference time of the Raspberry Pi 4 microcomputer, such that the operations execute within the minimum 2GB RAM and generate the specimen-level ovarian cancer subtype classification in under ten seconds per histopathological image file.
